(19) United States
(12) Patent Application Publication
(10) Pub. No.: US 2022/0287662 A1
(43) Pub. Date: Sep. 15, 2022

(54) X-RAY APPARATUS WITH STATIC TUBE HOLDER/ORTHOGONAL BUCKY WITH ISOCENTRICALLY ROTATING ARTICULATED ARM

(71) Applicant: VMI TECNOLOGIAS LTDA, Lagoa Santa (BR)

(72) Inventor: Otavio VIEGAS, Lagoa Santa (BR)

(21) Appl. No.: 17/597,933

(22) PCT Filed: Jul. 16, 2020
(86) PCT No.: PCT/BR2020/050269

§ 371 Date: Jan. 28, 2022

(30) Foreign Application Priority Data
Jul. 2, 2020 (BR) 10 2020 013650-0

(51) Int. Cl. A61B 6/00; A61B 6/04; A61B 6/02

(52) U.S. Cl. CPC A61B 6/4458; A61B 6/0407; A61B 6/4476; A61B 6/466; A61B 6/025

(57) ABSTRACT

The present invention, which addresses to the field of human and veterinary health, aiming at health security inspection, presents the creation of an unprecedented product, for the generation of radiographic images captured in 3D planar and multiplanar modes (Tomosynthesis) of the thorax, abdomen, skull, facial bones, upper/lower limbs, vertebral column, and the like, in routine and emergency examinations, and triage with the patient in the lying down, upright or sitting position, wherein the positioning movements are manual, motorized, or robotic, without restricting.

There are described in the present patent of innovation, for use in the field of human and animal health, the generation of radiographic images of the thorax, abdomen, skull, facial bones, upper and lower limbs, vertebral column, and the like, in routine and emergency examinations, and triage with the patient in the lying down, upright or sitting position. Positioning movements are manual, motorized, or robotic without restriction.

Since its discovery in 1895, the X-ray has become one of the most celebrated inventions in the world given its importance. Already in 1896, a year after its creation, it had resulted in the publication of 49 books and 1,000 scientific papers and some Nobel prizes to scientists and researchers dedicated to the subject. This interest of scholars focused on the subject persists until the present time, presenting constant evolutions and breaking of paradigms, always with contribution to the field of human and animal health.

The invention described in the present patent aims to provide the capture of images for clinical radiological studies with more angular precision, lower dose of radiation, greater productivity, lower production and maintenance costs and greater acuity, with a great capacity of perception of structures. In this way, the state of the art is innovated with a better technological solution to the problem of acuity in radiographs.

The innovative concept of the ‘Stative Tube Holder/Bucky Orthogonal with Isocentric Rotating Articulating Arm’ enables planar images conceived at angles with linear movements at 90 degrees (current technology) can be designed with uniform rotational motion and negative and positive (−/+) X-ray firing angles, with high accuracy.

It is important to highlight that stability, without vibrations, keeping the focal point without fluctuations allows three-dimensional images and super thin axial slices that enable the visualization of complex structures, such as the effects of COVID-19 on the lung in an early stage. This possibility acquires special importance in cases of a pandemic where the urgency and accuracy of the diagnosis means saving lives.

The equipment makes it possible to perform exams in vertical (Mural Bucky) and horizontal (Bucky Table) positions, both for conventional (2D) and tomosynthesis (3D) exams. The innovative design of the described assembly makes it possible for a large number of exams performed by computed tomography (CT) to be performed by this new equipment more quickly, with lower investment cost, and mainly, with much lower dose of radiation in the patient.

In searches carried out in the main patent banks, there have been found some similar technologies, identifying initial or more severe lung problems, but they do not present solutions similar to those proposed in this patent.

Patent CN109459861 presents an examination device for diagnosis by radiological imaging. The examination device comprises a shell, two parallel rotating rods and a motor system and gears to promote the rotation of the device during radiological imaging.

Patent US2019298288, in its turn, presents an analysis method to automatically determine radiological results data from radiology data records. The method comprises a neural network for analyzing the data records, an analysis unit and a control device used to control a diagnostic station.

Patent CN108549154 presents an adjustable radiology image viewing device. The device structure has wheels for moving, fixed and movable rods and a control panel for lifting and rotating the device, which makes it easier for clinicians to better observe a radiological image and make the diagnosis. However, such a device is not capable of generating radiological images, and is used only to facilitate the visualization of the images.

Patent US2017358079 presents a method for analyzing quantitative information obtained from radiological images. The method includes segmenting the radiological image and extracting quantitative features that allow to analyze the image. Thus, it is possible to create an image record, store the record in a data structure and generate a patient report with diagnosis.

Patent PI 0402727-2 presents a portable radiological equipment with a support device and a radiological diagnosis method. Such radiological equipment is capable of being easily transported between various locations, and is effective in treating patients who are unable to attend dental offices.

Patent BR1120170242141 presents a portable backscatter imaging inspection apparatus, an imaging method and a computer readable storage medium. The device comprises an X-ray source, a rotational modulation mechanism, a ray detector, a motion sensor and a controller.

In the present application for patent of invention, “X-RAY APPARATUS WITH STATIVE TUBE HOLDER/BUCKY ORTHOGONAL WITH ISOCENTRIC ROTATION ARTICULATED ARM”, there is described an unprecedented multipurpose X-ray product for use in hospitals, radiological services clinics, health centers, veterinary clinics, among others.

It is an invention of significant importance, especially in times of a pandemic, caused by new corona viruses, in which prompt and rapid action is required for its control. It is represented by an X-ray generator assembly, with specific technical features, and which has the following advantages:
Ability to generate uniplanar (2D) and multiplanar (3D) images.
Image generation using the multiplanar acquisition technique (MPR), which will allow the reconstruction of images for another viewing plane from a series.
High-precision isocentric rotation, which makes it possible to perform tomosynthesis exams in bucky table and bucky mural positions.
Bucky with motorized turn, coordinated with the holder/bucky arm, allowing the performance of different types of exams with the same equipment.
Availability of three-dimensional and two-dimensional images in a single X-ray machine.
Lower operating cost.
Smaller physical area for installing the entire assembly.
High productivity.
High spatial and density resolution that allows the detection of small lesions.
Lower dose of radiation.
Lower cost of the exam.
More assertive exams.

The figures below help to understand the proposed technology.

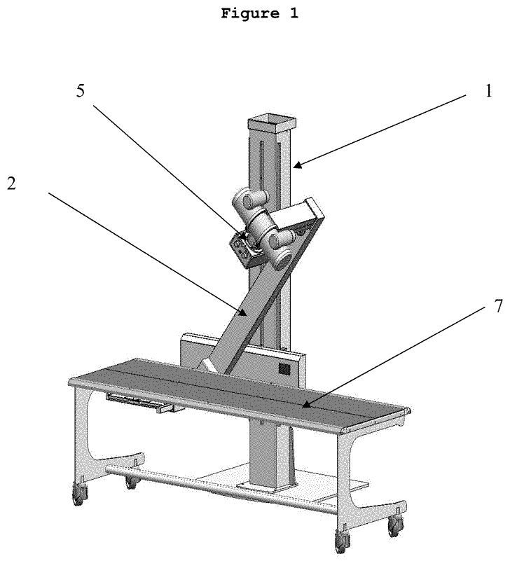

FIG. 1 presents a three-dimensional view of the complete equipment, including the examination table.

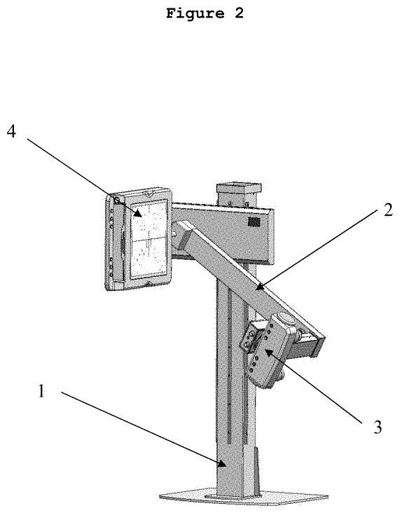

FIG. 2 presents a three-dimensional view of the multifunctional module of the stative tube holder/Bucky orthogonal with isocentric rotation articulated arm.

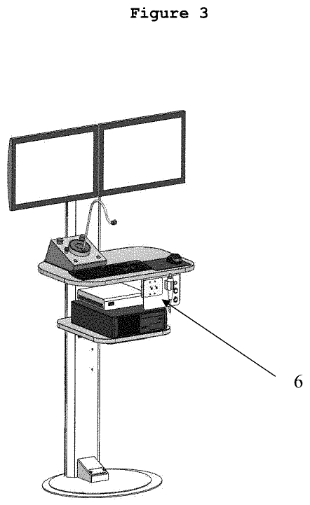

FIG. 3 shows a view of the digital image capture panel.

The invention described herein comprises a stative (1) (Vertical column) Tube holder/Bucky orthogonal with articulated arm (2) of isocentric rotation (turn), with vertical, longitudinal and/or rotational movements. The standard configuration includes an X-ray source (3), digital image capture detector (4), X-ray emitter (5), three-dimensional image reconstruction system (6) with computer, display and control software and examination table (7) on wheels, fixed or remote, without restriction.

Image capture occurs in both planar and multiplanar modes, which allows reconstructing images to another three-dimensional viewing plane from a series. Three-dimensional (3D) reconstruction is an analysis technique in which data from all axial slices are summarized into a few images, allowing TOMOSYNTHESIS (Synthesized Tomography) examination.

The listed advantages referring to the present invention are of absolute importance in the health system, both human and veterinary, and in particular, if moments of epidemics and even pandemics are taken into account, in which the abovementioned advantages can mean a quick solution to a diagnosis and the appropriate treatment for groups of people and/or animals.

The technical description of the equipment, which is the object of this patent, demonstrates the innovation brought to the state of the art and the importance of its components in the formation of the technological assembly described herein. It also demonstrates the relevance of the invention to the health field, preventing and providing accurate and agile diagnoses for the indication of appropriate treatments.

The STATIVE (1) TUBE HOLDER—ISOCENTRIC TURN equipment is intended to perform radiographic examinations on patients in the upright, sitting and lying down positions, respecting their physical conditions. It also has technical features of being able to be provided with manual, motorized and robotic motorized movement, without limitation.

THE STATIVE (1) TUBE/BUCKY HOLDER WITH ARTICULATED ARM (2) ISOCENTRIC ROTATION is a multifunctional module suitable for performing all routine radiographic examinations, including those of tomosynthesis and specialized areas such as trauma. It is worthy to highlight that the movement of the arm, tube holder in relation to the bucky and, in relation to the tube holder arm, and the perfect centralization of the focal point will provide the performance, with great precision, of numerous radiology exams.

In turn, the PIVOTING EXAMINATION TABLE (7) ON WHEELS refers to an examination table with a homogeneous, biocompatible, radio-transparent floating top and side rails, with top dimensions of about 74 cm (width)×225 cm (length), load capacity of 250 kg, load factor 4×=(1,000/kg). In the case of the table with pivoting turn, the dimensions of the top and load capacity are not pre-defined, but can be varied, if necessary.

The LUMINOUS COLLIMATOR (4) for adjusting the area to be irradiated, with flat blades for deep cutting, light field with radiated field centering indicators and rails for attaching accessories, is also characterized by a timed activation for the lamp and laser indicator, with (+180/−180°) Rotation, Inherent Filtration and 150 KV Protection.

The X-RAY EMITTER ASSEMBLY (5) consists of a 150 KV X-ray tube/300 KHu heat capacity or greater, with focal points around 0.6/1.2 mm. It has high anode rotation, with high voltage cables—150 KV insulation and high thermal capacity of the emitter assembly.

Its COMMAND/GENERATOR (3) is formed by a high frequency generator—microprocessor controls, with high power, resonant technology/switching by IGBTs, KV and MA adjustments, MAS adjustment, exposure time adjustment, PAO—(Anatomical organ program), PDA—(Meter of product×dose×area), indication of tube heating in KHu, safety protection—online, intelligent anode braking. It is further formed by independent command of preparation and firing and three-phase power supply 220 V-380 V-50/60 Hz.

It is up to the THREE-DIMENSIONAL IMAGE RECONSTRUCTION SYSTEM (6), which is composed of a digital image capture panel with an active area of 43×43, the transmission of data—wired or wireless, with a high resolution matrix, converter 14, 16 Bits, or greater, a preview in 2 (two) seconds—image formation in 7 (seven) seconds. For this function, it also has a computer, a capture software, available on the market for the medical field, reconstruction and distribution of images in two or three dimensions and a full HD display—Touch Screen.

1. An X-Ray apparatus with stative tube holder/bucky orthogonal with isocentric rotation articulated arm, comprising a Stative (Vertical Column), a tube holder arm/Bucky of isocentric rotation and vertical displacement, an X-ray generator assembly, a digital image detector, an X-ray emitter assembly, a three-dimensional image reconstruction system and an examination table.

2. The X-Ray apparatus with stative tube holder/bucky orthogonal with isocentric rotation articulated arm according to claim 1, wherein the displacement of the X-ray tube in relation to the Bucky (Angular movement) has a motorized turn and allows performing an isocentric rotation, being able to be used in vertical, horizontal and angled positions.

3. The X-Ray apparatus with stative tube holder/bucky orthogonal with isocentric rotation articulated arm according to claim 1, wherein the three-dimensional image reconstruction system comprises a digital image capture panel, a computer, a software for capturing, reconstructing and distributing images in two or three dimensions and a display that generates uniplanar and multiplanar images.

4. The X-Ray apparatus with stative tube holder/bucky orthogonal with isocentric rotation articulated arm according to claim 1, wherein tomosynthesis examinations (Synthesized Tomography) are carried out.

5. The X-Ray apparatus with stative tube holder/bucky orthogonal with isocentric rotation articulated arm according to claim 1, wherein an image is captured in fluoroscopy and multiplanar modes.
